(19) United States
(12) Patent Application Publication
(10) Pub. No.: US 2024/0173647 A1
(43) Pub. Date: May 30, 2024

(54) DEVICE AND METHOD FOR SIMULATED MOVING BED SEPARATION WITH A LARGE HEIGHT/DIAMETER RATIO

(71) Applicant: IFP ENERGIES NOUVELLES, Rueil-Malmaison (FR)

(72) Inventors: Alexandre VONNER, Rueil-Malmaison Cedex (FR); Damien LEINEKUGEL-LE-COCQ, Rueil-Malmaison Cedex (FR); Guillaume BLANCKE, Rueil-Malmaison Cedex (FR); Frédéric AUGIER, Rueil-Malmaison Cedex (FR); Aude ROYON-LEBEAUD, Rueil-Malmaison Cedex (FR); Manel FOURATI, Rueil-Malmaison Cedex (FR); Amir Hossein AHMADI-MOTLAGH, Rueil-Malmaison Cedex (FR)

(73) Assignee: IFP ENERGIES NOUVELLES, Rueil-Malmaison (FR)

(21) Appl. No.: 18/283,737

(22) PCT Filed: Mar. 15, 2022
(86) PCT No.: PCT/EP2022/056646

§ 371 Date: Sep. 22, 2023

(30) Foreign Application Priority Data
Mar. 25, 2021 (FR) FR2103022

(51) Int. Cl. B01D 15/18

(52) U.S. Cl. CPC B01D 15/1842

(57) ABSTRACT

The present invention relates to a device and a process for separation by simulated moving bed comprising a plurality of adsorbers (1, 2) which are positioned in series and arranged alternately for upflow and downflow, each adsorber (1, 2) being divided into n adsorption chambers each comprising one adsorbent bed, the n adsorbent beds being separated by n plates for injecting at least one feed and a desorbent and withdrawing at least one extract and a raffinate.

### TECHNICAL FIELD

The present invention relates to a device and a process for separation by simulated moving bed, notably for the separation of para-xylene from a mixture of aromatic compounds comprising 8 carbon atoms (ortho-xylene, meta-xylene and ethylbenzene).

### PRIOR ART

The current technologies for separation by simulated moving bed (sometimes abbreviated to SMB in the remainder of the text) use units which have a certain number of common features:
one or two adsorbers (or separating columns) each comprising a plurality of adsorption chambers positioned between a distribution duct and a collecting duct, said adsorption chambers comprising an adsorbent bed within which a fluid flows,
injection systems, notably for injecting the feed and the desorbent, and withdrawing systems, notably for withdrawing the produced effluents referred to as extract and raffinate,
collection and redistribution systems, referred to as inter-bed zones, for passing from one bed to the next bed.

A problem with the current technology for separation by SMB is that the adsorbers comprise a large number of beds (typically 12 or more beds) with a low height H to diameter D ratio (typically much lower than 1), in order that the adsorber is not too high and thus difficult to set up, use and maintain.

Another problem with the current technology for separation by SMB is that the flow of the fluids in the adsorbent beds is from top to bottom, the fluids from the bottom of the adsorber being raised to the top of the adsorber or of the following adsorber, which requires a substantial investment in terms of energy and equipment (valves, pumps, etc.) in order to push thousands of m³/h of fluid over a height of more than about ten metres.

Another problem with the current technology for separation by SMB is that the adsorbers comprise large volumes of the zones referred to as inter-bed zones, which limits the separation capacity and increases the need to use heavy equipment.

Another problem with the current technology for separation by SMB is that all the beds (e.g. 12 beds) of an adsorber need to be stopped in order to maintain or repair a predetermined section of the adsorber.

### SUMMARY OF THE INVENTION

In the context described above, a first object of the present description is to overcome the problems of the prior art and to provide a device and a process for separation by SMB which are simpler to set up, use and maintain.

A second object of the present description is to provide a device and a process for separation by SMB which allow savings to be made in terms of energy and equipment.

A third object of the present description is to provide a device and a process for separation by SMB which allow only some of the beds (e.g. one bed or a pair of beds) to be isolated without stopping the remainder of the device, which is able to operate in a fallback mode, with a satisfactory performance.

According to a first aspect of the invention, the above-mentioned objects, as well as other advantages, are obtained by a device for separation by simulated moving bed comprising a plurality of adsorbers which are positioned in series and arranged alternately for upflow and downflow, each adsorber being divided into n adsorption chambers each comprising one adsorbent bed, the n adsorbent beds being separated by n plates for injecting a feed and a desorbent and withdrawing an extract and a raffinate.

According to one or more embodiments, the number of adsorbers m is between 3 and 15, preferably between 4 and 12, and highly preferably between 5 and 10.

According to one or more embodiments, the number of adsorbers m is an even number.

According to one or more embodiments, the number n of adsorbent beds per adsorber is between 1 and 4, preferably between 1 and 3, and highly preferably between 1 and 2.

According to one or more embodiments, the total number t of adsorbent beds is between 6 and 24, preferably between 8 and 19, and highly preferably between 12 and 15.

According to one or more embodiments, the adsorbent beds have a height H to diameter D ratio H/D equal to or greater than 1, preferably equal to or greater than 1.5, and highly preferably equal to or greater than 2.

According to one or more embodiments, the adsorbent beds have a height H to diameter D ratio H/D between 1 and 12, preferably between 1.5 and 10, and highly preferably between 2 and 8.

According to one or more embodiments, the device further comprises at least one bypass line adapted to bypass at least one adsorber.

According to one or more embodiments, the bypass line is adapted to bypass two adjacent adsorbers.

According to one or more embodiments, the bypass line connects a downflow adsorber to an upflow adsorber.

According to one or more embodiments, the adsorbers are arranged in a vertical position for substantially vertical distribution of the fluid flowing through the adsorbers.

According to one or more embodiments, the adsorbers are arranged in a horizontal position for substantially horizontal distribution of the fluid flowing through the adsorbers. Advantageously, the flow (upflow/downflow) in the absorbers may takes place in the horizontal plane according to two opposite directions.

According to a second aspect of the invention, the above-mentioned objects, as well as other advantages, are obtained by a process for separation by simulated moving bed using the device for separation by simulated moving bed according to the first aspect, the process comprising the following steps:
the adsorbers are fed with at least one feed and a desorbent, and at least one extract and at least one raffinate are withdrawn from said adsorbers, the adsorbent beds being interconnected in a closed loop, the feed and withdrawal points in the adsorbers being shifted over the course of time by an amount corresponding to one adsorbent bed with a switching time and determining a plurality of operating zones of the device for separation by simulated moving bed, and notably the following main zones:
zone I for desorption of a product (of interest) to be separated (e.g. para-xylene) is between the injection of the desorbent D and the withdrawal of the extract E;
zone II for desorption of the isomers of the product to be separated is between the withdrawal of the extract E and the injection of the feed F;
zone III for adsorption of the product to be separated is between the injection of the feed F and the withdrawal of the raffinate R; and
zone IV is between the withdrawal of raffinate R and the injection of desorbent D.

According to one or more embodiments, the adsorbent beds are distributed in zones 1 to IV according to the following configurations referred to as a/b/c/d type configurations:
a is the number of beds in zone I;
b is the number of beds in zone 1l;
c is the number of beds in zone III; and
d is the number of beds in zone IV, and

a=(t*0.2)*(1±0.2);

b=(t*0.4)*(1±0.2);

c=(t*0.27)*(1±0.2); and

d=(t*0.13)*(1±0.2), and

in which t is a natural integer between 6 and 24, preferably between 8 and 15.

According to one or more embodiments, the adsorbent beds are distributed in zones 1 to IV according to the following configurations referred to as a/b/c/d type configurations:
a is the number of beds in zone I;
b is the number of beds in zone 1l;
c is the number of beds in zone III; and
d is the number of beds in zone IV, and

a=(t*0.17)*(1±0.2);

b=(t*0.42)*(1±0.2);

c=(t*0.25)*(1±0.2); and

d=(t*0.17)*(1±0.2), and

in which t is a natural integer between 6 and 24, preferably between 8 and 15.

According to one or more embodiments, the process comprises at least one of the following operating conditions:
the desorbent is chosen from the group consisting of one or more isomers of diethylbenzene and toluene;
the adsorbent used comprises/consists of a faujasite chosen from the group consisting of BaX, BaKX and BaLSX;
the feed is chosen from the group consisting of a mixture of essentially C8 aromatic compounds;
the temperature in the adsorbent beds is between 140° C. and 189° C.;
the pressure is controlled so that the liquid phase is maintained at all points of the device;
the switching time ST is between 20 seconds and 90 seconds; and
the average rate of flow between the beds is between 1000 and 5000 m³/h.

Other features and advantages of the invention according to the aforementioned aspects will become apparent on reading the following description and non-limiting exemplary embodiments, with reference to the appended figures described below.

### LIST OF FIGURES

FIG. 1 shows, in diagrammatic form, a reference device for separation by SMB, in which the adsorbers are downflow adsorbers.

FIG. 2 shows, in diagrammatic form, a device for separation by SMB according to one or more embodiments of the invention, in which the adsorbers are alternately downflow and upflow adsorbers.

### DESCRIPTION OF THE EMBODIMENTS

The invention relates to a device and a process for separation by simulated moving bed, notably for the separation of para-xylene.

Embodiments of the device and of the process according to the aforementioned aspects will now be described in detail. In the following detailed description, many specific details are disclosed in order to provide a deeper understanding of the process. However, it will be apparent to a person skilled in the art that the process can be performed without these specific details. In other cases, well-known characteristics have not been described in detail in order to avoid unnecessarily complicating the description.

In the present patent application, the term “to comprise” is synonymous with (means the same thing as) “to include” and “to contain”, and is inclusive or open and does not exclude other elements which are not stated. It is understood that the term “to comprise” includes the exclusive and closed term “to consist”. Moreover, in the present description, the terms “essentially” or “substantially” correspond to an approximation of ±5%, preferably of ±1%, very preferably of ±0.5%. For example, an effluent comprising essentially or consisting of compounds A corresponds to an effluent comprising at least 95% by weight of compounds A.

The present invention may be defined as a device or a unit comprising a plurality of adsorbers (or separating columns) positioned in series for the separation by simulated moving bed of compounds (e.g. xylenes), each adsorber being divided into n adsorption chambers each comprising one adsorbent bed, the n adsorbent beds being separated by n plates or inter-bed zones (i.e., n distribution zones and n collection zones), in which the adsorbers are arranged alternately for upflow and downflow. The distribution and collection zones comprise collection and redistribution systems for passing the fluid flowing through the adsorber from one bed to the next bed.

In the present patent application, the first distribution zone of the first adsorbent bed and the last collection zone of the last adsorbent bed are considered to form together a single inter-bed zone.

### The Device

A device for separation by simulated moving bed according to the invention is, for example, a chromatographic column or adsorption column, operating as a simulated moving bed. Separation by simulated moving bed is a well-known technique. As a general rule, the column operating as a simulated moving bed comprises at least three zones, generally four, and optionally five, each of these zones comprising a certain number of successive adsorbent beds (e.g. fixed beds), and each zone being defined by its position between a feed point and a withdrawal point. Typically, a simulated moving bed column is fed with at least one feed F (mixture of xylenes) to be fractionated and a desorbent D (sometimes referred to as eluent), and at least one raffinate R (mixture of xylenes depleted in para-xylene) and an extract E (desorbent and para-xylene) are withdrawn from said column. The feed and withdrawal points are modified over the course of time, typically shifted in the same direction by an amount corresponding to one adsorbent bed.

Advantageously, the alternating arrangement for upflow/downflow of the adsorbers of the device and of the process according to the present invention makes it possible to improve the construction of the unit, to simplify and limit the necessary equipment, to improve the separation of the fluids, and to reduce the energy costs for operation by reducing the volume of the zones referred to as inter-bed zones. Moreover, the present invention allows the unit to be maintained, with the possibility of totally isolating only one pair of beds without stopping the remainder of the unit, which is able to operate in a fallback mode, with a performance which is satisfactory although sometimes reduced.

The prior art describes in depth various devices that make it possible to achieve the separation of feeds by simulated moving bed. Particular mention may be made of U.S. Pat. Nos. 2,985,589, 3,214,247, 3,268,605, 3,592,612, 4,614,204, 4,378,292, 5,200,075, 5,316,821.

According to one or more embodiments, the distribution and collection zones comprise systems for injection, notably for injection of a feed (e.g. mixture of xylenes) and a desorbent (e.g. toluene or para-diethylbenzene), and withdrawal, notably of the produced effluents referred to as extract (e.g. mixture of para-xylene and desorbent) and raffinate (e.g. mixture of ortho-xylene, meta-xylene, desorbent and optionally ethylbenzene).

The controlled means/devices for feeding and withdrawing fluids to/from a device for separation by simulated moving bed are, for example, of one of the following two main types of technology:
either, for each plate, a plurality of controlled on/off valves (optionally with flow regulating elements) for feeding or withdrawing the fluids, these valves typically being situated in the immediate vicinity of the corresponding plate. Each plate typically comprises at least four two-way valves, controlled on an on/off basis, in order respectively to feed the feed and the desorbent and withdraw the extract and the raffinate;
or a multi-way rotary valve for feeding or withdrawing fluids across all of the plates.

The present invention notably falls within the context of columns operating in simulated moving bed mode that employ a plurality of valves for feeding and withdrawing the various fluids.

Preferably, the number m of adsorbers is between 3 and 15, preferably between 4 and 12, and highly preferably between 5 and 10. Preferably, the number m of adsorbers is an even number.

Preferably, the number n of adsorbent beds per adsorber is between 1 and 4, preferably between 1 and 3, and highly preferably between 1 and 2.

Preferably, the total number t of adsorbent beds is between 6 and 24, preferably between 8 and 19, and highly preferably between 12 and 15.

According to one or more embodiments of the present invention, the adsorbent beds have a height H to diameter D ratio H/D of at least 1 (preferably greater than 1), preferably of at least 1.5, and highly preferably of at least 2, such as at least 3 or 4.

According to one or more embodiments of the present invention, the adsorbent beds have a height H to diameter D ratio H/D between 1 and 12, preferably between 1.5 and 10, and highly preferably between 2 and 8.

An additional advantage of the device and of the process according to the invention is the possibility of using adsorbent beds having a greater height H to diameter D ratio H/D than in the prior art, which gives access to the use of radial beds, which permit improved separation.

According to one or more embodiments, the device according to the invention further comprises a bypass line adapted to bypass at least one adsorber, for example two adsorbers of the device. Advantageously, it is possible to isolate only some of the adsorbent beds for maintenance, for example by isolating one or two successive adsorbers, and to continue the separation process with a satisfactory performance which is improved compared with, for example, stopping an adsorber with 12 beds.

According to one or more embodiments, the bypass line connects an upstream adsorber i (i being between 1 and m) to a downstream adsorber i+2 or a downstream adsorber i+3. According to one or more embodiments, the bypass line is adapted to bypass, for example by means of a bypass valve, the adsorber i+1 and/or the adsorber i+2 by sending the streams leaving the adsorber i to the adsorber i+2 or the adsorber i+3. According to one or more embodiments, the bypass line connects a downflow adsorber i to an upflow adsorber i+3.

According to one or more embodiments, the adsorbers are arranged in a vertical arrangement. The invention also applies in the case of a horizontal arrangement of the adsorbers. The flow then takes place in the horizontal plane and in two opposite directions. The concept remains identical to the upflow/downflow, and the associated benefit is the same.

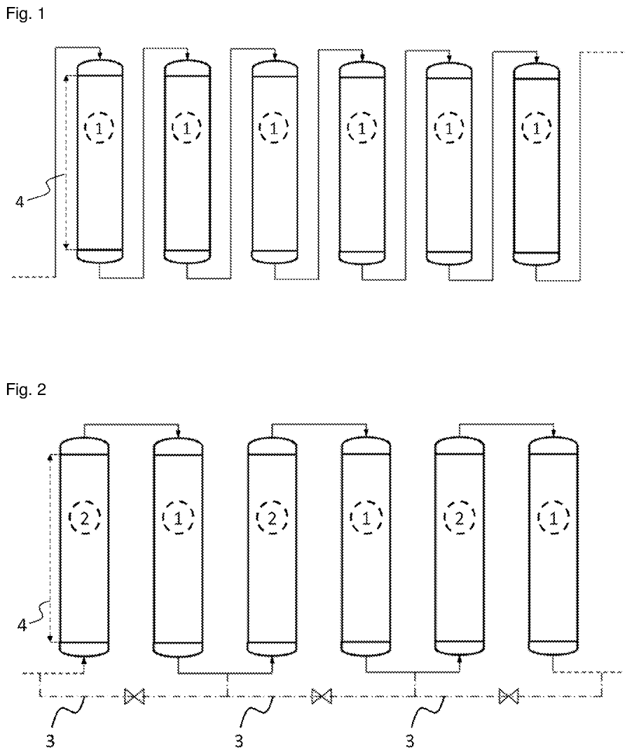

With reference to FIG. 1, a reference device for separation by SMB comprises a plurality of downflow adsorbers 1, each adsorber comprising a shell positioned between an upper dome and a lower dome. The downflow arrangement of the reference device corresponds to the flow of the fluids generally as a downflow in all the adsorbers by means of a line connecting the bottom of an adsorber i to the top of the next adsorber i+1. Generally, the reference devices are units with 24 beds comprising two downflow adsorbers each containing 12 beds.

With reference to FIG. 2, a device for separation by SMB according to one or more embodiments of the invention comprises a plurality of adsorbers arranged alternately for upflow and downflow, each adsorber comprising a shell positioned between an upper dome and a lower dome.

The alternating arrangement of the downflow adsorbers 1 and upflow adsorbers 2 of the device according to the invention corresponds to a flow of the fluids that is generally an upflow in adsorbers i, i+2, +4, etc. and a flow of the fluids that is generally a downflow in adsorbers i+1, +3, +5, etc., the top of an adsorber i being connected to the top of the next adsorber i+1 and the bottom of the adsorber i+1 being connected to the top of the next adsorber i+2. The device for separation by SMB according to one or more embodiments of the invention further comprises a plurality of bypass lines 3, each bypass line 3 connecting (in the direction of flow) an upstream downflow adsorber 1 to a downstream upflow adsorber 2, thus bypassing 2 adsorbers.

### The Process

In the remainder of the text, the term “step” is used to denote an operation or a group of similar operations carried out on a given stream at a certain point of the process. The process is described in its various steps taken in the order of flow of the streams or products.

The process for separation by SMB comprises the following steps: the adsorbers are fed with at least one feed F and a desorbent D, and at least one extract E and at least one raffinate R are withdrawn from said adsorbers, said adsorbers comprising one or more beds of an adsorbent solid which are interconnected in a closed loop (i.e. the last bed of the last adsorber being adapted to send the flowing stream to the first bed of the first adsorber), the feed and withdrawal points in the adsorbers being shifted over the course of time by a value corresponding to one adsorbent bed with a switching time (denoted ST) and determining a plurality of operating zones of the SMB device, and notably the following main zones:

By definition, each of the operating zones is denoted by a number:
zone I for desorption of the para-xylene is between the injection of the desorbent D and the withdrawal of the extract E;
zone II for desorption of the isomers is between the withdrawal of the extract E and the injection of the feed F;
zone III for adsorption of the para-xylene is between the injection of the feed F and the withdrawal of the raffinate R; and
zone IV is between the withdrawal of raffinate R and the injection of desorbent D.

According to one or more embodiments, the adsorbent beds are distributed in zones I to IV according to configurations referred to as a/b/c/d type configurations, that is to say that the distribution of the beds is the following:
a is the number of beds in zone I;
b is the number of beds in zone 1l;
c is the number of beds in zone III; and
d is the number of beds in zone IV.

According to one or more embodiments:

a=(t*0.2)*(1±0.2);

b=(t*0.4)*(1±0.2);

c=(t*0.27)*(1±0.2); and

d=(t*0.13)*(1±0.2), and

in which t is a natural integer between 6 and 24, preferably between 8 and 15 (e.g. 12).

According to one or more embodiments:

a=(t*0.17)*(1±0.2);

b=(t*0.42)*(1±0.2);

c=(t*0.25)*(1±0.2); and

d=(t*0.17)*(1±0.2), and

in which t is a natural integer between 6 and 24, preferably between 8 and 15 (e.g. 12).

In normal operation (for example, without bypassing an adsorber), the feed and the withdrawal are carried out with a predetermined proportion of the number of adsorbent beds operating in zones I to IV. For example, the proportion of adsorbent beds in zone I in normal operation is equal to a/t, wherein t is the total number of adsorbent beds. According to one or more embodiments, when a number x of adsorbers is bypassed, the controlled devices for feeding and withdrawing (e.g. system with multiple all-or-nothing valves, multi-way rotary valve) are adapted to modify the position of the feed and withdrawal points so that the predetermined proportion of the average number of adsorbent beds operating in zones I to IV is maintained within an approximation of ±20%, preferably ±10%, very preferably ±5%. For example, the proportion of adsorbent beds in zone I during bypassing operation is equal to a/(t-x).

In the present description, a zone comprising “an average number” of adsorbent beds corresponds to a zone which may punctually comprise a first (natural) number X of adsorbent beds, and punctually a second (natural) number X−1 or X+1 of adsorbent beds. For example, a zone I comprising 2.5 beds, punctually includes 2 adsorbent beds and punctually includes 3 adsorbent beds.

According to one or more embodiments, during a first part of the permutation period ST (e.g. when permutations of the feed and withdrawal points are out of phase), at least one zone comprises a first number X of adsorbent beds; and during a second part of the permutation period ST, the at least one zone comprises a second number X+1 or X−1 of adsorbent beds.

According to one or more embodiments, during a first cycle time, or a first part of a cycle time, at least one zone comprises a first number X of adsorbent beds; and during a second cycle time, or a second part of the cycle time, the at least one zone comprises a second number X+1 or X−1 of adsorbent beds. In the present description, the cycle time corresponds to the time during which the injection and withdrawal points of the device evolve until they return to their initial position.

According to one or more embodiments, when a number x of adsorbers is bypassed, the feed and the withdrawal are carried out during bypassing operation according to the following configuration a/b/c/d:

a=((t−x)*0.2)*(1±0.2);

b=((t−x)*0.4)*(1±0.2);

c=((t−x)*0.27)*(1±0.2); and

d=((t−x)*0.13)*(1±0.2), and

in which t is a natural integer between 6 and 24, preferably between 8 and 15 (e.g. 12).

According to one or more embodiments, when a number x of adsorbers is bypassed, the feed and the withdrawal are carried out during bypassing operation according to the following configuration a/b/c/d:

a=((t−x)*0.17)*(1±0.2);

b=((t−x)*0.42)*(1±0.2);

c=((t−x)*0.25)*(1±0.2); and

d=((t−x)*0.17)*(1±0.2), and

in which t is a natural integer between 6 and 24, preferably between 8 and 15 (e.g. 12).

Preferably, the number x of bypassed adsorbers is equal to 1 or 2, more preferably, x is equal to 2.

Let us consider for example a reference simulated moving bed separation device, such as a tower comprising 12 beds stacked in the same shell for the separation of products, such as xylenes. In the event of a bed malfunction (leak, breakage, etc.) on a 12-bed tower, the entire tower should be stopped, unloaded and reloaded before restarting. This involves long periods when the device is shut down and there is no production.

Let us now consider a device according to the invention, comprising for example 12 adsorbers for the separation of products, such as xylenes, in which a bed is defective. The bed can be isolated using the bypass line, preferably with an adjacent bed. The bypass is preferably done from the bottom of the adsorbers, thus making it possible to open the heads of the insulated adsorbers to unload and repair them. During the maintenance operation, the simulated mobile bed can continue to operate with the remaining 10 beds.

Advantageously, it is possible to adjust the sequence so as to maintain the same distribution of adsorbent beds or sieves by zones. For example, for a normal operation in configuration 3/6/4/2. The maintenance can be performed during a bypassing operation in the 2.6/5.2/3.5/1.7 configuration. Advantageously, the process according to the present invention may implement a synchronous movement of the inlet/outlet pipes, but may also implement a non-synchronous movement of the inlet/outlet valves in the device according to the present invention, a non-synchronous movement being also known as VARICOL. Advantageously, production may be maintained at the desired purity. The yield of the operation may be somewhat affected by a lower volume of available sieves.

According to one or more embodiments, the desorbent is chosen from the group consisting of one or more isomers of diethylbenzene and toluene. According to one or more embodiments, the desorbent is para-diethylbenzene or toluene. According to one or more embodiments, the desorbent is toluene.

According to one or more embodiments, the adsorbent used comprises/consists of a faujasite chosen from the group consisting of BaX, BaKX and BaLSX.

According to one or more embodiments, the feed is chosen from the group consisting of a mixture of essentially C8 aromatic compounds (e.g. xylenes and ethylbenzene). According to one or more embodiments, the mixture comprises at least 95%, preferably at least 97% (e.g. at least 99%) of essentially C8 aromatic compounds. According to one or more embodiments, the feed comprises at least 15 wt % of para-xylene and/or 30 wt % of meta-xylene relative to the total weight of the feed.

One example of an SMB separation process of great industrial importance is the separation of C8 aromatic fractions in order to produce para-xylene of commercial purity, typically at a purity of at least 99.7 wt %, and a raffinate rich in ethylbenzene, ortho-xylene and meta-xylene.

The extract produced contains desorbent, para-xylene and optionally traces of isomers (para-xylene purity of greater than 95%, preferably greater than 98%). This extract can be treated in order to separate the desorbent (e.g. by distillation) and then purified either by crystallization or by adsorption by simulated moving bed in order to increase the purity of the para-xylene.

According to one or more embodiments, the temperature in the adsorbent beds is between 140° C. and 189° C. and preferably between 155° C. and 185° C., particularly preferably between 170° C. and 180° C.

The pressure is adjusted so that the liquid phase is maintained at all points of the process according to the invention. According to one or more embodiments, the pressure in the adsorbent beds is between 1 MPa and 10 MPa, preferably between 2 MPa and 4 MPa, more preferably between 2 MPa and 3 MPa.

According to one or more embodiments, the switching time ST (time between two successive switchings of the feeds/extractions) used is between 20 seconds and 90 seconds. Preferably, the switching time ST used is between 30 seconds and 70 seconds (e.g. 50±10 seconds).

According to one or more embodiments, the average rate of flow between the beds is between 1000 m³/h and 5000 m³/h, preferably between 2000 m³/h or 2500 m³/h and 4000 m³/h, and highly preferably between 3000 m³/h and 4000 m³/h.

### EXAMPLES

### Reference Device

Consider a reference device for separation by SMB consisting of 12 adsorbers, each containing 1 adsorbent bed with a volume of 45.9 m³ and a height H to diameter D ratio H/D of 4.

The flow is a downflow in each adsorber.

The diameter of each adsorber is 3 m, the height 4 between the tangent lines of the domes is 12 m.

The average rate of flow between the beds is 3850 m³/h.

The inter-bed volume of the unit is estimated to be 177.7 m³.

### Device According to the Invention

Consider a device for separation by SMB according to the invention consisting of 12 adsorbers, each containing 1 adsorbent bed with a volume of 45.9 m³ and a height H to diameter D ratio H/D of 4.

The flow is alternately an upflow and a downflow in the successive adsorbers.

The diameter of each adsorber is 3 m, the height 4 between the tangent lines of the domes is 12 m.

The average rate of flow between the beds is 3500 m³/h.

The inter-bed volume of the unit is estimated to be 125.2 m³. This example notably illustrates the gain in inter-bed volume, which results in a gain in terms of the rate of flow in the unit and therefore an operational gain in terms of the operating cost as well as the cost of constructing the unit (reduced mass of metal).

1. Device for separation by simulated moving bed comprising a plurality of adsorbers (1, 2) which are positioned in series and arranged alternately for upflow and downflow, each adsorber (1, 2) being divided into n adsorption chambers each comprising one adsorbent bed, the n adsorbent beds being separated by n plates for injecting a feed and a desorbent and withdrawing an extract and a raffinate.

2. Device according to claim 1, wherein the number of adsorbers m is between 3 and 15.

3. Device according to claim 1, wherein the number of adsorbers m is an even number.

4. Device according to claim 1, wherein the number n of adsorbent beds per adsorber (1, 2) is between 1 and 4, preferably between 1 and 3, and highly preferably between 1 and 2.

5. Device according to claim 1, wherein the total number t of adsorbent beds is between 6 and 24, preferably between 8 and 19, and highly preferably between 12 and 15.

6. Device according to claim 1, wherein the adsorbent beds have a height H to diameter D ratio H/D equal to or greater than 1, preferably equal to or greater than 1.5, and highly preferably equal to or greater than 2.

7. Device according to claim 1, wherein the adsorbent beds have a height H to diameter D ratio H/D between 1 and 12, preferably between 1.5 and 10, and highly preferably between 2 and 8.

8. Device according to claim 1, further comprising at least one bypass line (3) adapted to bypass at least one adsorber (1, 2).

9. Device according to claim 8, wherein the bypass line (3) is adapted to bypass two adjacent adsorbers (1, 2).

10. Device according to claim 8, wherein the bypass line (3) connects a downflow adsorber (1) to an upflow adsorber (2).

11. Device according to claim 1, wherein the adsorbers (1, 2) are arranged in a vertical position for substantially vertical distribution of the fluid flowing through the adsorbers (1, 2).

12. Device according to claim 1, wherein the adsorbers (1, 2) are arranged in a horizontal position for substantially horizontal distribution of the fluid flowing through the adsorbers (1, 2), the flow through the adsorbers (1, 2) taking place in a horizontal plane and in two opposite directions.

13. Process for separation by simulated moving bed using the device for separation by simulated moving bed according to claim 1 any one of the preceding claims, the process comprising the following steps:
the adsorbers (1, 2) are fed with at least one feed and a desorbent, and at least one extract and at least one raffinate are withdrawn from said adsorbers (1, 2), the adsorbent beds being interconnected in a closed loop, the feed and withdrawal points in the adsorbers (1, 2) being shifted over the course of time by an amount corresponding to one adsorbent bed with a switching time and determining a plurality of operating zones of the device for separation by simulated moving bed, including the following main zones:
zone I for desorption of a product to be separated is between the injection of the desorbent D and the withdrawal of the extract E;
zone II for desorption of the isomers of the product to be separated is between the withdrawal of the extract E and the injection of the feed F;
zone III for adsorption of the product to be separated is between the injection of the feed F and the withdrawal of the raffinate R; and
zone IV is between the withdrawal of raffinate R and the injection of desorbent D.

14. Process according to claim 13, wherein the adsorbent beds are distributed in zones I to IV according to the following configurations referred to as a/b/c/d type configurations:
a is the number of beds in zone I;
b is the number of beds in zone II;
c is the number of beds in zone III; and
d is the number of beds in zone IV, and
a=(t*0.2)*(1±0.2);
b=(t*0.4)*(1±0.2);
c=(t*0.27)*(1±0.2); and
d=(t*0.13)*(1±0.2), and
in which t is a natural integer between 6 and 24, preferably between 8 and 15.

15. Process according to claim 13, wherein the adsorbent beds are distributed in zones I to IV according to the following configurations referred to as a/b/c/d type configurations:
a is the number of beds in zone I;
b is the number of beds in zone II;
c is the number of beds in zone III; and
d is the number of beds in zone IV, and
a=(t*0.17)*(1±0.2);
b=(t*0.42)*(1±0.2);
c=(t*0.25)*(1±0.2); and
d=(t*0.17)*(1±0.2),and
in which t is a natural integer between 6 and 24, preferably between 8 and 15.
